(19) United States
(12) Patent Application Publication
(10) Pub. No.: US 2011/0137201 A1
(43) Pub. Date: Jun. 9, 2011

(54) ORAL THERMOMETER WITH CURVED PROBE

(76) Inventor: Jacob Fraden, San Diego, CA (US)

(21) Appl. No.: 12/922,607

(22) PCT Filed: Jul. 23, 2010
(86) PCT No.: PCT/US10/43053

§ 371 Date: Jan. 13, 2011

Related U.S. Application Data
(60) Provisional application No. 61/271,541, filed on Jul. 23, 2009.

(51) Int. Cl. A61B 5/01

(52) U.S. Cl. 600/549

(57) ABSTRACT

An oral thermometer including a probe for insertion into the mouth of a user. The probe includes and an elongate first stem, a joint stem extending the first stem and disposed at a first angle to the first stem, and an elongate second stem extending from the joint stem and disposed at a second angle to the joint stem. The first angle open down and the second angle open up. The second stem includes a probe tip at a distal end thereof that is configured to be inserted into the mouth of a user. In operation the thermometer is operable to heat the probe tip to a pre-warmed temperature, detect an elevated temperature higher than the pre-warmed temperature when inserted in a user's mouth, and initiate a temperature reading upon detecting the elevated temperature.

### FIELD OF INVENTION

This invention relates to devices for measuring temperature, and specifically relates to thermometers primarily intended for medical applications.

### DESCRIPTION OF PRIOR ART

A contact medical thermometer is a device capable of measuring temperature through physical contact with the object of measurement. Typically, the probe of an oral thermometer is placed inside the patient's mouth in a sublingual pocket. The measurement is done by detecting the response of a temperature sensor that is built into the probe.

Oral thermometers are well known and their designs range from the glass tube filled with liquid as exemplified by U.S. Pat. No. 3,780,586 issued to Donofrio, to liquid crystal probes as exemplified by U.S. Pat. No. 4,779,995 issued to Santacaterina et al, to a plastic tube with a metal sensing tip as exemplified by U.S. Pat. No. 4,813,790 issued to Frankel et al.

To improve the thermal coupling between the temperature sensitive probe and the sublingual pocket tissues, a resilient pacifier probe has been proposed as exemplified by U.S. Pat. No. 5,176,704 issued to Bernd and a flexible probe as described by U.S. Pat. No. 5,013,161 issued to Zaragoza et al. A stem of the probe may be given a permanent bend to facilitate a better thermal contact with the patient's lip as taught by U.S. Pat. No. 7,036,984 issued to Penney et al.

Response speed is a major issue with any contact thermometer and specifically with oral thermometers. When a colder (e.g., room temperature) probe is placed in the patient's mouth, it alters the oral tissue temperature so much that a substantial time is required to re-warm the oral tissue to a pre-insertion level. Typically, this time may range from 6 seconds to a minute. If the re-warming time is ignored, accuracy may be compromised. One way to minimize this thermal drag by a cooler probe is to pre- warm the probe to a temperature that is substantially close to the oral anticipated temperature. This approach is exemplified by U.S. Pat. No. 5,632,555 issued to Gregory et al. and U.S. Pat. No. 6,109,784 issued to Weiss.

Due to talking and breathing, many spots in the mouth, even in a sublingual pocket, may have lower temperatures than that of the inner (core) body. A preferred place for the oral temperature measurement is an area near the root of the tongue in the sublingual pocket which is well shielded from the outside. This area has a more consistent and stable temperature.

Conventional probes have several drawbacks, such as poor coupling between the probe and the root of the tongue which reduces accuracy and prolongs the measurement time. Another limitation of conventional heated probes is the need for a manual initiation of measurement upon inserting the probe into the patient's mouth. Yet another limitation is the ease of placing the probe in the wrong spot inside the mouth by an inexperienced operator.

Thus, there is a need for an oral probe design that facilitates an intuitive self-guidance toward the root of the tongue when placed in the mouth. Moreover, there is a need for a probe that increases thermal contact between the probe body and the tissue of interest, has a fast response speed and requires only limited control by the operator.

### SUMMARY OF INVENTION

In an embodiment, the present invention provides an oral temperature probe having a shape that is sculptured to facilitate self-guidance toward the root of the tongue. The probe body has at least two bends in opposite directions so it resembles a letter Z. This shape allows the probe to curve around the teeth and direct the sensing tip toward the root of the tongue. The shape of the tip may be formed such that the area contacting the tissue is larger than the side area of the tip that is not intended for touching the tissue. The probe tip may be preheated to a temperature that is cooler than the lowest expected temperature of the patient and the measurement cycle may be initiated when the tip temperature approaches the lowest expected temperature of a patient.

### BRIEF DESCRIPTION OF DRAWINGS

Exemplary embodiments of the present invention are described in more detail below with reference to the drawings, in which:

FIG. 1 is a representative view of a medical oral thermometer inserted into a sublingual pocket;

FIG. 2 shows a view of the probe having two bends;

FIG. 3 illustrates a cross-sectional view of the probe tip with the temperature sensor and heater; and

FIG. 4 is a timing diagram of the thermometer operation.

### DESCRIPTION OF PREFERRED EMBODIMENT

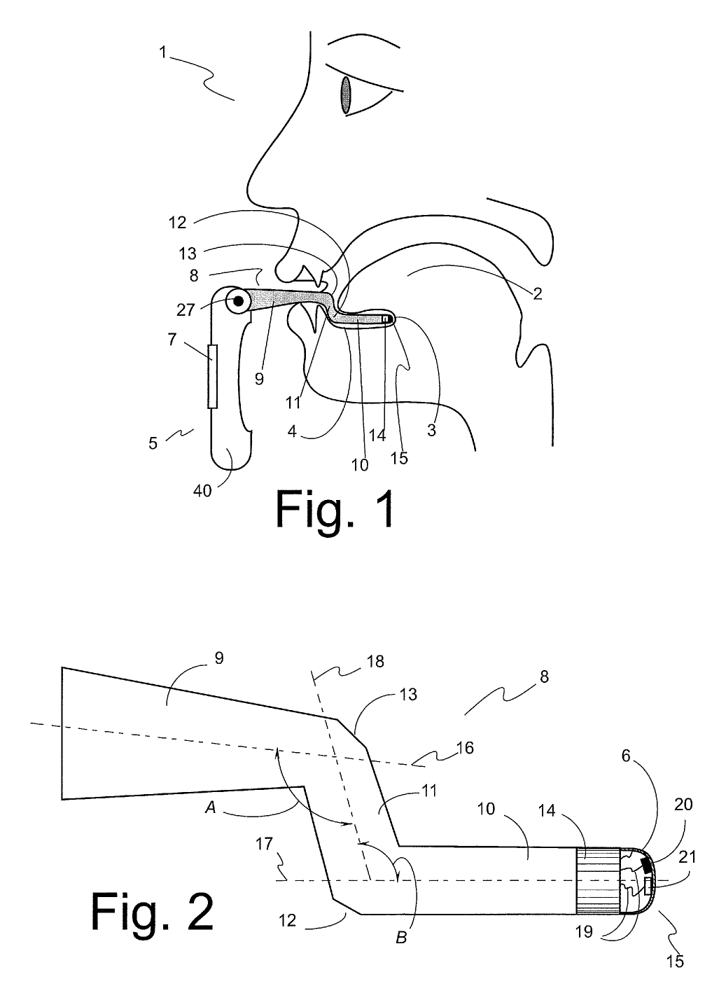

FIG. 1 illustrates a probe 8 of an oral thermometer 5 inserted into a sublingual pocket 4 of patient 1. The probe tip 15 makes thermal contact with the tongue root 3. The thermometer 5 inside or on its housing 40 contains an output element 7 that may be a visual display, audio speaker, wired or wireless transmitter, etc. The output temperature is the result of a signal processing performed by an electronic circuit and software residing inside the thermometer housing 40. The probe 8 is sculptured or formed in a Z-shape having the first stem 9, second stem 10 and joint stem 11. The Z-shape is facilitated by two bends in the probe: first bend 13 and second bend 12. The tip 15 may be separated from the second stem 10 by a thermal insulator 14. This thermal insulator may be needed when the second stem 10 has higher thermal conductivity, for example, when it is fabricated of metal. During the measurement, the second stem 10 is positioned under the tongue 2 in such a manner as to make reliable contact with the tongue root 3. Often, when measuring oral temperatures, patients place probes randomly in the oral cavity, either missing the sublingual pocket 4 or not pressing the probe tip against the tongue root. The Z-shape allows for an intuitive placement of the probe 8 inside the sublingual pocket 4 with the tip being forced to touch the tongue root. Any other position of the probe will likely be uncomfortable and thus, intuitively avoidable.

FIG. 2 depicts probe 8 with two bends 13 and 12 formed in the opposite directions. This creates a Z-shape of the probe. The first, second and joint stems (9, 10 and 11, respectively) may be the hollow tubes of any suitable cross-section, such as round, oval, etc. Each stem has its own axis. That is, the first stem 9 is disposed along the first axis 16, the second stem 10 is disposed along the second axis 17, while the joint stem 11 is disposed along the joint axis 18. These three axes sequentially cross each other, wherein the first and joint axes make an angle A, while the joint and second axes make an angle B. In an embodiment, each of the angles A and B may range from 45 to 135 degrees. Typically, the second stem 10 may have a length (along second axis 17) of about 25 mm, but no less than 10 mm. This allows positioning the second stem 10 behind the teeth and creates a reliable thermal contact between the tip 15 and the tongue root 3 for most patients. The second stem 10 may be fabricated either from a rigid or flexible/resilient material, so it will be able to accommodate variations in a distance between the patient's teeth and the tongue root 3. The area where the first stem 9 is attached to the housing 40 of the thermometer 5 may be made as a pivot 27, allowing for the probe 8 to rotate toward the case 40 during storage and away from the case 40 during operation. Inside the case 40, there may be an electric switch that signals the electronic circuit on the rotation (closing and opening) of probe 8, so that electric power may be turned off and on accordingly.

The joint stem 11 may have a length (along second axis 18) of about 15 mm to accommodate for the height of human teeth and gums, but the length is typically no less than 8 mm. The length of the first stem 9 may be any suitable length, depending of a particular thermometer design. The first and joint stems 9 and 11 may be fabricated of any suitable rigid material. A low thermal conductivity plastic may be used for the second stem 10. However, if the second stem 10 is fabricated of a material having relatively high thermal conductivity, a low thermal conductivity (thermal insulator) insert 14 can be positioned between the second stem 10 and the tip 15.

The tip 15 may be fabricated with a metal cup 6. Inside the cup 6, temperature sensor 20 is positioned. Sensor 20 can be of any suitable nature, such as a thermistor, thermocouple, RTD, etc. For a higher speed response, the tip 15 also may contain a heater 21. The sensor 20, heater 21 and the cup 6 are connected to the electronic circuit by conductors 19. The cup 6 may be gold plated.

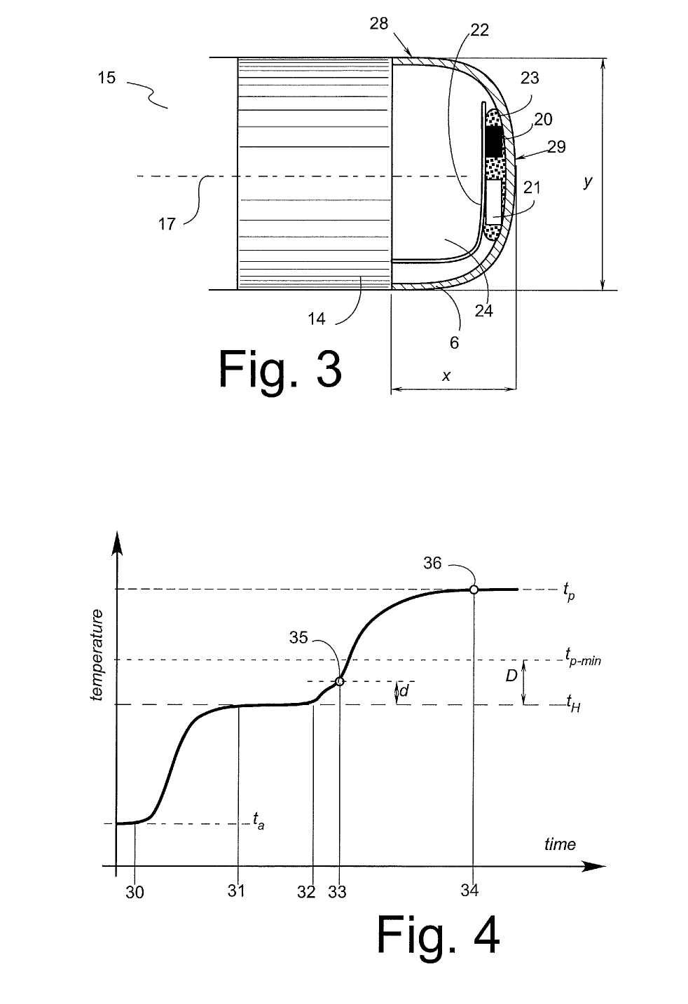

FIG. 3 shows an embodiment of the tip 15 with a flexible strip 22 that carries the sensor 20 and, possibly, heater 21. The strip 22 also carries the electrical conductors. The strip 22, sensor 20 and heater 21 may be attached to the inner surface of the cup 6 by a thermally conductive epoxy 23. Thus, the cup 6, sensor 20 and heater 21 will be in an intimate thermal coupling with each other. Besides, these components, the inner space 24 of the cup 6 may be void of any material (with a possible exception of air), thus a thermal coupling between the sensor 20 and other components positioned outside the cup 6 will be minimized. The cup 6 has side walls 28 disposed substantially parallel to the second axis 17 and the end wall 29 being substantially perpendicular to the second axis 17. The end wall 29 is intended for contacting the measured tissue. Even if the cup has a hemi-spherical shape, the corresponding tangents to the surface (side and end) are situated in the above described manner. It should be noted that the area of the end wall 29 can be as large as practical for an acceptable response speed, while side 28 area may be minimized. Hence, the length x (along the second axis 17) should be minimized while the dimension y (normal to the second axis 17) should be maximized. For most practical cases, length x may be between 1 and 3 mm, while dimension y may be 4 mm or larger.

To assure ease of operation and fast speed response, heater 21 may be turned on/off in a prescribed manner. Also, a signal produced by the sensor 20 can be processed in relationship with the heater operation. FIG. 4 illustrates an embodiment of the relationships between various temperatures of the cup 6 and thermometer actions. At first, the cup 6 has initial temperature tₐ which may be room temperature. Patient oral temperature in a sublingual pocket is tₚ, while the lowest possible temperature of the sublingual pocket is tₚ₋ₘᵢₙ. For example, it may be assumed that the patient particular temperature is tₚ32 39.4° C., while the lowest oral temperature of any human subject is tₚ₋ₘᵢₙ=34° C.

At first time instant 30, the thermometer is turned on and the electronic control circuit supplies electric energy to the heater to elevate its temperature to a level of pre-warmed temperature t_{H} that is lower than the lowest patient temperature tₚ₋ₘᵢₙ=34° C. For example, t_{H}=33° C. For most practical purposes, the difference D between these two temperatures can be at least 0.5° C. When the sensor 20 reaches the pre-warmed temperature t_{H} at the second time instant 31, this temperature is maintained by the feedback loop of the electronic circuit for as long as needed to place the probe into the mouth of the patient.

The operator places the probe into the patient's mouth and at the third time instant 32, the cup 6 contacts the tongue root 3 which quickly elevates the sensor 20 temperature above the t_{H} level. This “jump” in temperature is detected by the electronic circuit when its value 35 reaches a preset threshold at the fourth time instant 33. It should be noted that the jump threshold value (t_{H}+d) should be less than or equal to expected tₚ₋ₘᵢₙ.

At this fourth time instant 33, the heater 21 is turned off and temperature of the cup 6 is allowed to continue rising up to the patient temperature tₚ, which is reached at the fifth time instant 34 when the cup temperature has the end value 36. At this fifth time instant 34 the cup 6 and the tongue root 3 are in a thermal equilibrium, the measurement is over and the end value 36 temperature is provided by the output element 7. Since the time interval between time instants 33 and 34 is much shorter than the time interval between time instants 30 and 34, the time from placing the probe in the mouth (32) and the end of measurement at the fifth time instant 33 is drastically reduced. Typically, it is less than three seconds.

One aspect of this invention is that the pre-warmed temperature t_{H} is cooler than the patient's minimum temperature (t_{H}<tₚ₋ₘᵢₙ) and the measurement cycle is initiated when the tip temperature approaches the patient's minimum temperature tₚ₋ₘᵢₙ. This allows for an automatic detection of the probe placement in the mouth and thus eliminates a need for a manual control of the temperature taking cycle.

In cases when the initial temperature tₐ is already warm, that is it is equal or higher than the patient minimum temperature tₚ₋ₘᵢₙ, the heater is never turned on and the cup 6 allowed to equilibrate with the patient tₚ temperature, just as in conventional equilibrium thermometers.

In other embodiments, the heater 21 is not employed and no probe pre-warming performed. In such an embodiment, the measurement time is either accepted as being slower or it may be shortened by using one of several known predictive algorithms.

The invention has been described in connection with a preferred embodiment, but the invention is greater than and not intended to be limited to the particular form set forth. The invention is intended to cover such alternatives, modifications, and equivalents as may be included within the spirit and scope of the invention as defined by the appended claims.

All patents and other documents referred to herein are incorporated by reference in their entirety.

What is claimed is:

1. An oral thermometer comprising:
a probe configured to be at least partially inserted into the mouth of a user, the probe including:
an elongated first stem having a distal end and a proximal end;
a joint stem having a distal end and a proximal end, the joint stem extending from the distal end of the first stem and disposed at a first angle to the first stem about a first bend; and
an elongated second stem having a distal end and a proximal end, the second stem extending from the distal end of the joint stem and disposed at a second angle to the joint stem about a second bend,
wherein the first angle opens generally in a downward direction and the second angle opens generally in an upward direction.

2. The oral thermometer of claim 1 further comprising a temperature sensor disposed at the probe tip.

3. The oral thermometer of claim 1 further comprising a heater operable to heat the probe tip.

4. The oral thermometer of claim 1 wherein each of the first and second angle is between 45 and 135 degrees.

5. The oral thermometer of claim 2 wherein the probe tip includes a metal cup and wherein the temperature sensor is disposed inside the metal cup.

6. The oral thermometer of claim 1 wherein the second stem has a length of at least 10 mm.

7. The oral themiometer of claim 6 wherein the second stem has a length of about 25 mm.

8. The oral thermometer of claim 1 wherein the joint stem has a length of at least 8 mm.

9. The oral the mometer of claim 8 wherein the joint stem has a length of about 15 mm.

10. The oral thermometer of claim 1 further comprising a housing, wherein the probe is pivotally attached to the housing.

11. The oral theuiiometer of claim 10 further comprising a switch operable to activate the thermometer in response to pivoting between the probe and the housing.

12. The oral thermometer of claim 3 further comprising an electronic control unit operable to heat the probe tip to a pre-warmed temperature t_{H} that is lower than a lowest patient temperature tₚ₋ₘᵢₙ;
detect an elevated temperature higher than the pre-warmed temperature by a predetermined threshold using a temperature sensor disposed in proximity to the probe tip; and
initiate a temperature reading upon detecting the elevated temperature.

13. The oral thermometer of claim 12 wherein the electronic control unit is further operable to maintain the probe tip at the pre-warmed temperature for at least a set amount of time until the elevated temperature is detected.

14. The oral thermometer of claim 12 wherein the electronic control unit is further operable to turn off the heater in response to the probe tip reaching the elevated temperature.

15. The oral thermometer of claim 1 further comprising a low thermal conductivity insert disposed between the second bend and the probe tip.

16. The oral thermometer of claim 1 wherein the probe is generally z-shaped.

17. A method of measuring a temperature of a user with an oral thermometer comprising:
providing a thermometer with a probe having a first stem with a distal end and a proximal end, a joint stem with a distal end and a proximal end, the joint stem extending from the distal end of the first stem and disposed at a first end angle to the first stem, and a second stem with a distal end and a proximal end extending from the distal end of the joint stem and disposed at a second angle to the joint stem, wherein the first angle opens generally in a downward direction and the second angle opens generally in an upward direction;
heating a probe tip of the probe to a pre-warmed temperature t_{H} that is lower than a lowest patient temperature tₚ₋ₘᵢₙ using a heater disposed in a proximity of the probe tip;
inserting the probe into a mouth of the user;
detecting an elevated temperature higher than the pre-warmed temperature by a predetermined threshold using a sensor disposed in the proximity of the probe tip; and
initiating a temperature reading upon detecting the elevated temperature.

18. The method of claim 17 further comprising maintaining the probe tip at the pre-warmed temperature for at least a set amount of time until the elevated temperature is detected.

19. The method of claim 17 further comprising turning off the heater in response to the probe tip reaching the elevated temperature.

20. An oral thermometer comprising:
a probe configured to be at least partially inserted into the mouth of a user, the probe including:
an elongated first stem having a distal end and a proximal end;
a joint stem having a distal end and a proximal end, the joint stem extending from the distal end of the first stem and disposed at a first angle to the first stem about a first bend; and
an elongated second stem having a distal end and a proximal end, the second stem extending from the distal end of the joint stem and disposed at a second angle to the joint stem about a second bend,
wherein the first stem and second stem extend from the joint stem in substantially opposite directions.
